(19) United States
(12) Patent Application Publication
(10) Pub. No.: US 2023/0060119 A9
(43) Pub. Date: Feb. 23, 2023

(54) Wearable Tension Apparatus

(71) Applicant: Alignmed, Inc., Santa Ana, CA (US)

(72) Inventors: William J. Schultz, Newport Beach, CA (US); Michael Morganti, Castro Valley, CA (US)

(21) Appl. No.: 17/228,595

(22) Filed: Apr. 12, 2021

Related U.S. Application Data
(63) Continuation of application No. 17/177,109, filed on Feb. 16, 2021.
Application No. 17/177,109 is a continuation of application No. PCT/US20/14844, filed on Jan. 23, 2020.
Application No. PCT/US20/14844 is a continuation of application No. PCT/US20/13970, filed on Jan. 17, 2020.
Application No. PCT/US20/13970 is a continuation of application No. PCT/US19/55740, filed on Oct. 10, 2019.
Application No. PCT/US19/55740 is a continuation of application No. PCT/US19/46430, filed on Aug. 13, 2019.
Application No. 17/177,109 is a continuation of application No. PCT/US20/32753, filed on May 13, 2020.
Application No. 17/177,109 is a continuation of application No. PCT/US19/46430, filed on Aug. 13, 2019.
(60) Provisional application No. 62/796,035, filed on Jan. 23, 2019.
(60) Provisional application No. 62/847,210, filed on May 13, 2019.
(60) Provisional application No. 62/718,350, filed on Aug. 13, 2018.
(60) Provisional application No. 62/718,353, filed on Aug. 13, 2018.
(60) Provisional application No. 62/744,002, filed on Oct. 10, 2018.

(51) Int. Cl. A61F 5/02

(52) U.S. Cl. CPC A61F 5/026

(57) ABSTRACT

Exemplary embodiments contained herein include garments having biomechanical components including neurobands, posture mounts, expansion panels, and combinations thereof.

### BACKGROUND

From birth to death, we are all given one body to live in and help us perform our best. Unfortunately, a wide range of musculoskeletal, neurological and circulatory diseases and disorders exist as a matter of injury, environment, occupation, genetics and disease. A dynamic anatomic garment would provide an interface of therapeutic support and resistance to a body in motion, and thus intervene in the physical maladies associated with injury, arthritis, chronic pain, sensory processing disorder, muscle imbalance, joint misalignment and poor posture.

Current upper body performance apparel uses compression of skin and muscle, simultaneously or in gradients, as the active mechanism. Compressing skin and muscle does not offer an optimal means to train, retrain and maintain muscle balance or postural abnormalities.

In general, garments are worn by persons for different purposes including protection from environmental elements, modesty, adornment and fashion. Some garments are worn to support, or to influence neuromuscular activity for therapeutic results, or to improve physical position. An example is a form-fit garment, worn to aid muscle balance, body alignment, circulation and/or postural fitness. A form-fit garment may be used as an adjunctive treatment for chronic pain, injury, disease and disorder.

Shoulder, neck and back pain affect tens of millions of Americans every year, and result in significant disability, dysfunction, and time off work. Poor postures that develop over time as a result of environmental factors or injury are common causes of muscle and joint pain in the upper body. In general, garments are worn by persons for different purposes including protection from environmental elements, modesty, adornment and fashion. Some garments are worn to support and dynamically train musculoskeletal anatomy, with built in tensile resistance to influence the sensory systems and neuromuscular physiology for therapeutic results, or to improve physical position for improvements in performance, posture and physical appeal. An example is an anatomic, form-fit garment, worn to intervene, influence or otherwise aid the process of body balance, alignment, circulation and/or postural misalignment which may predispose a person to arthritis, pain and injury. A form-fit garment may be used as an adjunctive treatment for chronic pain, injury, disease and disorder.

Prophylactic muscle and joint braces are used by many people, from athletes to those with arthritis and injury, to mechanically restrict or otherwise alter movement and body position.

Taping techniques have been used for many generations to provide an exterior support and stability to muscles and joints to assist and facilitate the body's natural healing processes. Techniques similar to taping have been applied to exterior garments worn by a user. These garments provide a close fitting apparel that have bands or restrictive mechanisms integrated therein and/or in which separate bands are applied thereto. The apparel provides a base to attach or secure the bands so that contact or adhesion to the skin is not necessary. However, the idea is the same, the bands are configured to applied support and/or pressure to the body by attaching bands at different points on the body.

When using apparel as an intermediary to taping or applying pressure to or pull on body parts, the obvious drawback is the movement of the apparel relative to the body. For example, if a fitted shirt is used to attach one or more bands, the shirt anchoring locations may be pulled or relocated under the pressure imposed by the bands. Not only are the locations affected at the attachment points, but other portions of the apparel are also adversely influenced and generally pulled out of alignment. One of the most uncomfortable distortion in an underlying garment in in the armpit where the fabric of the garment may pull and bunch around the sensitive skin of the underarm.

In addition or alternatively thereto, most apparel or taping solutions provide linear applications of pull on the body. However, the underlying body mechanics are not so restrictive. Most appendages and muscle groups permit body movement in circular or spherical orbits. Linear apparel configurations may therefore impose uncomfortable pulling on a body part or a muscle group.

### SUMMARY

Exemplary embodiments of the wearable tension apparatus described herein may include an active mechanism including anatomic arrangements of elastic panels, bands and seams, collectively referred to as neurobands. Neurobands may apply tensile resistance to activate muscles in a kinetic sequence. Thus, resistance on specific muscles may be according to exemplary methods described herein to train, retrain and maintain otherwise activate muscle tissue. This therapeutic approach is unique to use tensile resistance to targeted muscles through a wearable tension garment. An exemplary advantage of such a wearable tension apparatus is to provide a passive therapy which requires no discipline or change in behavior to have measurable effects, outside of periodically wearing a comfortable, form-fit garment over or under clothing—even during sleep. The interface of neurobands with a body in motion provides and extraordinary way to harvest the energy of movement and thus get more out of your body—over suitable periods of time. As such, neurobands may serve as an adjunctive treatment in caregiver strategies for physical rehabilitation, pain management and injury prevention. In occupational environments where repetitive, awkward movements cause injury over time, the garments are designed to be worn comfortably over clothing, as a convenient and economical bio-ergonomic intervention to correspond with the needs of overused and underused muscle groups.

Exemplary embodiments described herein include garments worn by persons for different purposes including protection from environmental elements, modesty, adornment, fashion, support, influence neuromuscular activity for therapeutic results, or improve physical orientation. An example is a form-fit garment, worn to aid muscle balance, body alignment, circulation and/or postural fitness. A form-fit garment may be used as an adjunctive treatment for chronic pain, injury, disease and disorder.

Exemplary embodiments relate generally to a garment or device worn by a person for functional improvements in postural form, health and fitness, comfort, range of motion, reduced interference with range of motion, and combinations thereof.

Exemplary embodiments described herein include different design shapes for garments. The garments may combine comfortable, form-fitting posture garments for the upper and lower extremity or that traverse the upper and lower extremity. The posture garments may have built in attachment points which are anatomically positioned to accept panels of various elastic properties, shapes, tensile resistance and tactile feel. The panels may be attached to the garment or integrated therein.

Exemplary embodiments may include a wearable device, such as a garment, defining a non-linear and/or non-orthogonal construction of anatomic apparel portions used to systematically interact and influence the innate processes of human physiology for improvement in health and fitness.

Exemplary embodiments may comprise dynamic materials with specific elastic resistance, collectively referred to as neurobands. Neurobands may be placed in an interactive matrix to influence the force and direction of body movement and sensory information sent to and from the spinal cord and brain. Thus, a normalization of function can be achieved by reversing or slowing down dysfunctional adaptations that muscles and joints take on with time and injury.

Exemplary methods described herein may use neurobands to apply dynamic tensile resistance and tactile touch to prevent or reduce muscle atrophy by actively enforcing natural movement and neutral anatomy.

Exemplary embodiments of garments described herein provide for the seamless transfer of forces from a front of the garment to the back of the garment. For example, a garment for use on an upper body may have a seamless transition from the front of the garment to the back of the garment over a shoulder portion of the wearer when in an in use configuration on a wearer.

Exemplary embodiments described herein incorporate non-linear apparel construction. For example, embodiments described herein may be created by patterns and/or panels within linear portions or edges. Embodiments described herein may include patterns and/or panels without any linear portions or edges. Embodiments described herein may include patterns and/or panels in which edges configured to attach to another portion or panel consists of only curved, non-linear edges. In an exemplary embodiment, panels may be attached to avoid or minimize perpendicular seams, perpendicular panels, or perpendicular patterned pieces.

Exemplary embodiments described herein may comprise materials having a systematic property of elasticity including single and/or multi-directional orientations. In an exemplary embodiment, materials are knit or woven in an oblique orientation to avoid or reduce perpendicular alignment of the fibers. Exemplary embodiments comprise a warp weave with or without a weft.

Exemplary embodiments orient material panels such that a direction of a bias of stretch are oriented with respect to and/or aligned with a portion of the underlying muscle group for which the material is intended to overlay when in a worn position on a wearer. Exemplary embodiments include configurations, orientations, and material configured to mimic the natural underlying physiologic properties of the neuromuscular movement of the wearer. Exemplary embodiments include panels, orientations, configurations, and materials to create anatomic anchoring points such that portions of the garment correspond to underlying anatomic areas to provide a natural anchor of the garment relative to the body of the user when in a worn position.

In an exemplary embodiment, a garment may include a front panel with greater stretch to permit the expansion or variation of abdominal areas of a user. In an exemplary embodiment, the bias of stretch of the front portion of the garment configured to overlay the abdominal section of the wearer is in a horizontal or across the body direction. In an exemplary embodiment, the bias of stretch may be configured such that the garment is configured to stretch approximately or at least 25% more in the direction of the bias of stretch in the horizontal direction than in other directions. Other bias of stretch described herein may include a similar or the same 25% bias of stretch factor.

### DRAWINGS

FIG. 1A illustrates an exemplary garment according to embodiments described herein.

FIG. 1B illustrates the garment of FIG. 1A overlayed with the underlying muscle groups of an exemplary wearer in an in use position.

FIG. 2 illustrates an exemplary neuroband configuration according to embodiments described herein.

FIGS. 3A-3B illustrate an exemplary garment according to embodiments described herein.

FIGS. 4A-4B illustrate an exemplary garment according to embodiments described herein.

FIGS. 5A-5B illustrates an exemplary garment according to embodiments described herein.

FIGS. 6A-6C illustrates an exemplary garment according to embodiments described herein.

FIG. 7 illustrates an exemplary feature that may be incorporated into any garment described herein.

FIG. 8 illustrates an exemplary modular neuroband according to embodiments described herein.

FIGS. 9A-9C illustrates an exemplary neuroband configuration for use as a half top.

### DETAILED DESCRIPTION

In the following description of preferred embodiments, reference is made to the accompanying drawings which form a part hereof, and in which it is shown by way of illustration specific embodiments in which the invention can be practiced. It is to be understood that other embodiments can be used and structural changes can be made without departing from the scope of the embodiments of this invention.

As stated above, the symptoms of a wide variety of human conditions in which muscles, joints, sensory and circulatory systems are compromised, could be helped with an exoskeletal intervention that conforms to the unique environments in which we work and play. The wearable tension apparatus described herein may include a lifestyle technology designed to be comfortable, worn systematically, under or over clothing, over convenient periods of time. Wearable tension-technology is aimed to impact the sociologic and economic burden of musculoskeletal health.

Exemplary embodiments of the wearable tension apparatus described herein includes a garment that fits snugly on the individuals upper body anatomy using conformable elastomeric textiles with specific elastic memory (neurobands) and non-linear construction to address the distinct movements of a human body in both static and dynamic motion. Neurobands provide a multidirectional level of elastic force that is adjoined with a corresponding level of elastic force. Pending on the wearer's condition, and level of physiologic need, neurobands can be altered anteriorly, posteriorly, medially, laterally, superior or distal to anatomically correspond to the targeted muscles, joints and spinal vertebrae causing the wearer's symptoms. This would then result in localized resistance to train, retrain or maintain afflicted anatomy, thereby resulting in symptom reduction.

Current compression garments do not localize therapeutic resistance to a specific and precise level and consequently do not offer optimal training of over overused and underused muscles. Current compression garments and performance apparel target athletic of physically fit demographics due to the vanity of a form-fit and the difficulty in comfortably fitting anatomy with excessive weight and mass. In addition, the compressing of fat tissue (adipose tissue), does not address a means to reduce dysfunctional adaptions associated with obesity, such as poor postures, early fatigue, muscle imbalance, joint misalignment and compromised circulation. By having high ratios of elastane materials in the stomach, axila (armpit) and pectorals, and non-linear construction techniques throughout the garment, a conformable rate of expansion is greatly improved.

Exemplary embodiments of the wearable tension apparatus described herein is designed to prevent or reduce the severity of muscle and joint stress that result from injury, recurring injury or from the imbalances of overused and underused muscle groups that promote injury.

Neurobands described herein may produce kinetic support and tensile resistance onto targeted muscle tissue for the purpose of training and maintaining muscle balance, joint alignment and good posture. Tensile resistance is considered a standard of care in physical therapy and routinely used to improve performance, recover from or prevent an injury. Kinetic Millimeters of Force (KMF) is the exoskeletal force generated by the anatomic position of panels, bands and seams and textiles with specific elastomeric force. Exemplary embodiments of the wearable tension apparatus described herein include apparel having variable elasticity and elastic memory properties, placed onto specific anatomy. Such construction and placement are different than garments that use compression or gradient compression. Muscles contract in order to perform movement, they don't compress. Muscles contract in a kinetic sequence to optimize performance. Muscle tissue is extremely sensitive to tensile resistance and the position of a body within a constant field of gravity. To externally influence a muscle contraction requires an intervention of tensile resistance, or a repositioning of the body in space, otherwise referred to as an improvement in posture. Good improvement in posture is a very accurate gauge of health, fitness and physical appeal. It is not logical or likely that increasing pressure on skin and muscle, in the form of compression, will influence an improvement in muscle performance.

Exemplary embodiments of neurobands described herein induce kinetic support and tensile resistance in vertical, horizontal and oblique plains of motion. A greater convenience of exemplary embodiments of the wearable tension apparatus described herein including neurobands is that they provide therapy that does not require conscious discipline or change in behavior to be effective. The only conscious requirement is to periodically wear a form-fitting garment. A primary function of neurobands is displayed by the elastic force difference between two or more adjoining panels, bands and seams.

Thermogenesis is the fundamental process of heat production in muscles. The more active muscles are, the more thermogenesis they produce. The human body uses heat from thermogenesis as energy to be expended for dynamic (exercise) and static (sleeping, sitting) activity. Because the function of muscles relies on heat produced by tensile resistance in a field of gravity, multiplying the tensile resistance of a garment, for the purpose of converting heat as a passive modality to train, retrain and maintain muscle balance may be a benefit of neurobands described herein. The acceleration of body movement, may increase the tensile resistance and thermogenesis in doses that are purposeful to the wearer. A functional utility of an elastomeric panel, band or seam requires correction of overused and underused muscles for the purposed of improving muscle balance, joint alignment and posture and reducing the pain and inflammatory process associated with poor muscle balance and misaligned joints.

Sometimes the neurobands, including panels, bands, and seams vary in location and density to account for the amount of therapeutic force required. The elastomeric properties of each panel, band and seam can be described as having precise anatomic and physiologic purpose.

Exemplary embodiments described herein use neurobands. Exemplary embodiments may include procedures and techniques that use bands of variable stretch characteristics to mirror the innate physiology of the related muscle to which the band is positioned over. Exemplary embodiments described herein are in terms of bands, but the invention is not limited to a specific geometric configuration of “band”. Instead, any configuration of a material portion having the desired relative stretch characteristics and/or positions relative to the body are encompassed within the definition of “band”. Accordingly, bands may include linear or elongated pieces, circular pieces, and any combination of geometric or non-geometric portions as described herein or would be understood by a person of skill in the art.

In an exemplary embodiment, the described procedures and techniques may include positioning and orienting neurobands such that they are anchored to start and end in line with or over corresponding neuromuscular anatomical features. The configuration and position may therefore mirror or correspond to neuromuscular kinesiology or neuromuscular mapping. Exemplary embodiments of neurobands may be used to physically facilitate muscles and their function to support, load, and unload joints. Exemplary embodiments may be used to position joints in certain positions that are in line with good posture and optimal body mechanics. Exemplary embodiments may be used to enhance posture, reduce pain, and aid in recovery.

Exemplary embodiments described herein include a wearable device. The wearable device may use non-restrictive, anatomically fit, elastomeric (stretchable) materials configured and attached together corresponding to anatomic anchoring locations, and having material fiber orientations in-line with contractual properties of the muscles where the material sections overlay or link together, and combinations thereof. When worn, exemplary embodiments of a garment having features described herein may be used to interact with the shape and motion of the wearer to generate and/or deliver therapeutic modalities including, but not limited to, tactile biofeedback, muscle support, tensile resistance used to train, retrain, maintain, or otherwise improve the wearer's muscle balance, circulation, physical appeal and overall postural fitness. Exemplary embodiments may be used for muscle and postural training when worn intermittently.

Exemplary embodiments may be used with garments having a form fit, such as performance apparel, compression apparel, shapewear, maternal wear, among others. Such exoskeletal apparel may produce atypical tensions and pressures which can be restrictive, ill-fitting, ill-positioned, or uncomfortable. Exemplary embodiments may be used to improve anatomic conformability using construction techniques and materials that address the innate properties of human anatomy and physiology. Exemplary embodiments may use, configure, and position materials and component panels to synergistically assist or correspond with contractual properties of the muscle groups in which they overlay, contact, or traverse. Exemplary embodiments may configure material sections to define or correspond to an anatomical form having boarders and/or seams corresponding to anatomical anchoring locations to address the kinetic sequence of the muscle tissue they adjoin.

Exemplary embodiments may incorporate stretch and tensile characteristics that are placed directionally and in line with human kinetics. This may be done using a variety of anchor points on one or more form-fitting posture garments. Neurobands can link the upper and/or lower extremity using one or more garments.

Exemplary embodiments include using fabric fiber orientation and bias of stretch within a material as a mechanism of action to provide neurobio feedback to the wearer. The use of fiber orientation within exemplary embodiments may provide a precision based application of tensile resistance when placed in the kinetic direction of muscle force.

Exemplary embodiments include a garment and method that actively influences the body's own resources to preserve and maintain optimal alignment, that uses biofeedback to enhance the senses of proprioception, kinesthesia, touch and other components of the central, peripheral and somatosensory nerves to maintain and preserve good posture, that uses biofeedback to support optimal body mechanics while simultaneously enhancing muscle memory and muscle retraining, and combinations thereof.

Exemplary embodiments may include neurobands. Exemplary neurobands may include synthetic and/or natural materials with stretch characteristics designed to mimic the contractual properties of muscle tissue for which the neuroband overlays. Exemplary neuroband construction and fiber orientation can be adjusted to assist specific muscles and joints to perform specific functions. Exemplary neurobands may be constructed to allow their forces to be spread over a broad area. Exemplary neurobands may include tensile and tactile properties configured to dynamically provide biofeedback to facilitate muscle and joints movement in the performance of human mobility. Exemplary neurobands may be kinetically placed and may be adjusted to assist muscles and joints in performing specific functions.

Exemplary neurobands can be used to attain an optimal fit by adjustment in the banding and at the attachment site. Exemplary neurobands may be categorized based upon the exact arrangement between fibers and/or upon the respective pull on muscle tissue. Exemplary neurobands can be more uniform in diameter with essentially all fibers arranged in a long parallel manner or a cross section diameter depending on the muscle tissue it addresses and its ability to exert force and the corresponding muscle ability to shorten through range of motion.

Exemplary embodiments may include posture mounts to create attachment points for exemplary neurobands according to embodiments described herein. Posture mounts may be configured to provide anchor points to support the tensile (i.e. pull) function of neurobands according to embodiments described herein. Posture mounts may correspond or overlay tendon and/or ligament locations when the garment is positioned on the wearer. Therefore, posture mounts may provide specific anatomic locations to act as start and stop locations for associated neurobands that overlay corresponding muscle areas of the body. Posture mount attachment may be configured to assist muscle and joints to perform specific functions when one or more neurobands are attached thereto. Posture mounts may define rigid or semi-rigid mounting locations to assist and support attached neurobands.

Exemplary embodiments may include garments constructed to avoid compression in one or more areas of the body. For example, exemplary garments may include expansion panels configured to permit less restrictive movement or reduce pressure on the underlying anatomic features of the body. Exemplary expansion panels may be included over or around the abdomen, neck, thorax, armpit, other soft tissue areas, or combination thereof. Exemplary expansion panels may include bi-directional or multi-directional stretch orientations to provide greater expansion of the panel over select areas of the body.

Exemplary embodiments of a garment include exemplary seam constructions to coordinate specific elasticity and tensile support throughout the garment. Exemplary seam constructions and placement may correspond to human kinetics and directional transfer of tensile strength of associated neurobands to which the seam may be coupled.

Exemplary embodiments may include a garment according to embodiments described herein. Exemplary garments may include different portions or component parts to define neurobands and/or posture mounts according to embodiments described herein. Exemplary embodiments may include material portions having unidirectional, bidirectional, multi-directional bias of elasticity. Exemplary embodiments may orient the bias of elasticity in specific configuration and orientations to support the underlying body anatomy according to embodiments described herein. Exemplary embodiments may include posture mount locations in which the elasticity of the material is reduced compared to the neurobands such that posture mount locations may position and anchor the neurobands to locations relative to the underlying anatomy of the body. Exemplary embodiments may include form fitting garments having a front portion, a back portion, and arm portions. The exemplary garment may include lateral side portions that are incorporated into the front and/or back portions or may be separate thereto.

As used herein, elasticity generally refers to the amount a material can stretch under a given force. Therefore, a greater elasticity indicates a greater amount of stretch when a force is imposed thereon. Rigid or semi-rigid materials are considered to have low elasticity and do not stretch or only include a very limited stretch under an imposition of force. As used herein, the bias of stretch generally indicates that the material has a greater elasticity in the direction of the bias of stretch than in other directions. A material may have one or more than one bias of stretch. Each direction of a material's bias of stretch may have the same elasticity or different elasticities. Therefore, a material may have a major bias of stretch in a first direction and a second bias of stretch in a second direction, where the first bias of stretch has a greater elasticity than the elasticity associated with the second bias of stretch.

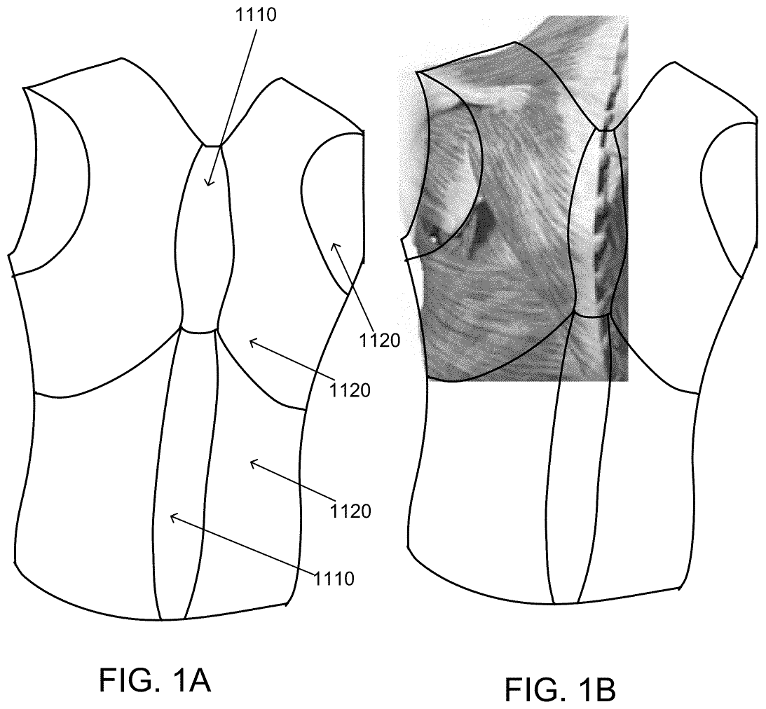

FIG. 1A illustrates an exemplary back side of a garment according to embodiments described herein made up of a plurality of neurobands 1120 and posture mounts 1110. FIG. 1B illustrates an exemplary illustration of the garment of FIG. 1A overlaid upon an exemplary muscle groups to illustrate the corresponding muscle tissue for which the neuroband overlays. The posture mounts 1110 may comprise separate panels or may be created from an overlap of adjacent neurobands, such that the posture mount provide a more rigid material support than the adjacent neurobands that may have a higher elasticity. The exemplary garment may include neurobands 1120 according to embodiments described herein directed over the shoulder of the wearer and toward and around a lateral side of the wearer. The exemplary neurbands 1120 approximates and corresponds to the muscle groups trapezius muscle and latissimus dorsi muscle of the wearer when positioned in a worn position over a body of the wearer. The exemplary posture mounts correspond to areas overlaying the spine of the wearer when positioned in a worn position over a body of the wearer.

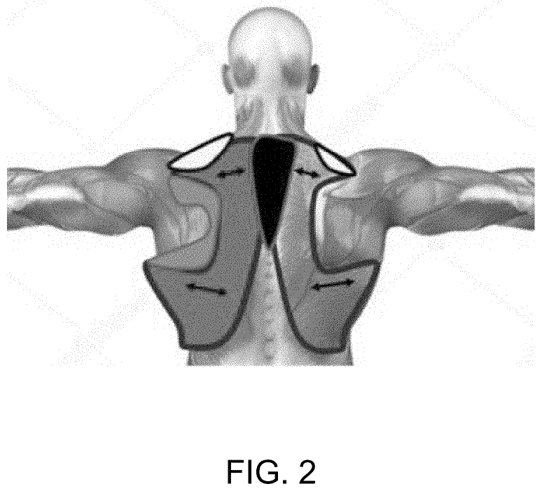

FIG. 2 illustrates an exemplary neuroband configuration according to embodiments described herein. A neuroband may be configured to be positioned over the trapezius muscle and latissimus dorsi muscle of a wearer when positioned in an in use position. A pair of neurobands may be configured in mirrored arrangement to be positioned on respective sides of the spine of the wearer. In an exemplary embodiment, the neuroband may define a generally curved upward seam that corresponds to an upper portion of the trapezius muscle and run along a top shoulder portion of a wearer. The neuroband may cross the spine of a wearer onto the opposing side of the body and then progress downward toward the waste of the wearer. On the downward, spine edge of the neuroband, the neuroband may cross the spine approximate to a middle of the back adjacent or between the shoulder blades or at a lower portion of the shoulder blades. A gap may be made and/or filled with a posture mount, or another neuroband between the neurobands illustrated in FIG. 2. The exemplary neuroband may wrap around an abdominal section of the wearer and up under the arm of the wearer. The exemplary embodiment of the neuroband may include an indentation corresponding to a shoulder blade or teres minor and/or teres major of the user. In an exemplary embodiment the bias of stretch of the neuroband of FIG. 2 is generally radially outward from a center of the back, generally horizontal when in a worn position, perpendicular to the longitudinal axis or axis of symmetry of the back portion, oblique to the longitudinal axis or axis of symmetry of the back portion, or combinations thereof. In an exemplary embodiment, two neurobands according to FIG. 2 are positioned in mirrored positions about the axis of symmetry of the back portion of the garment. The two neurobands may overlap at an upper, center portion overlaying a spine of the wearer in the worn position.

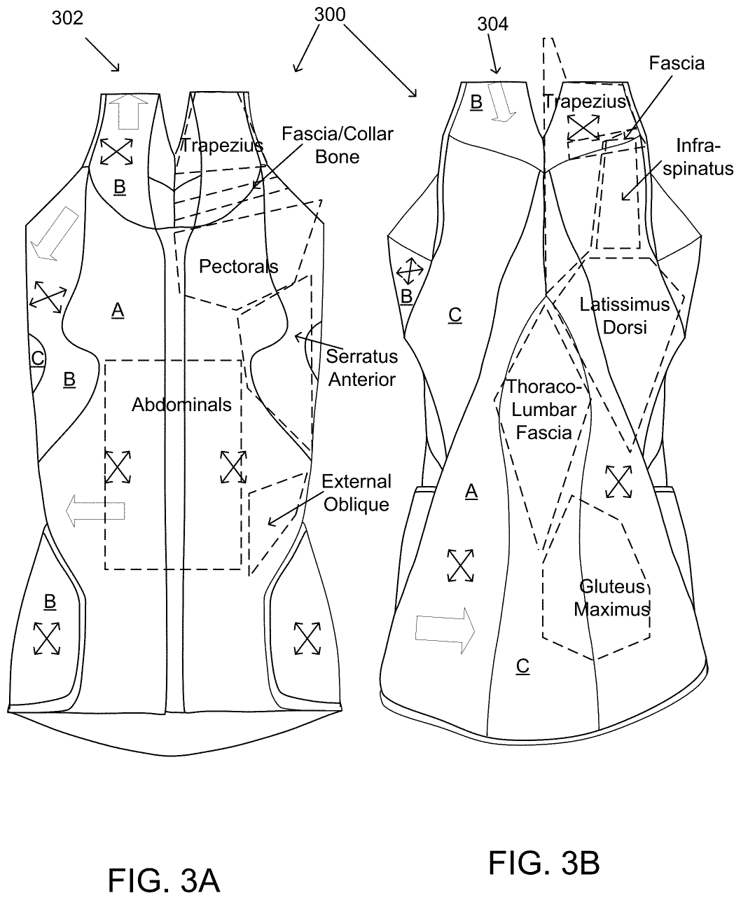

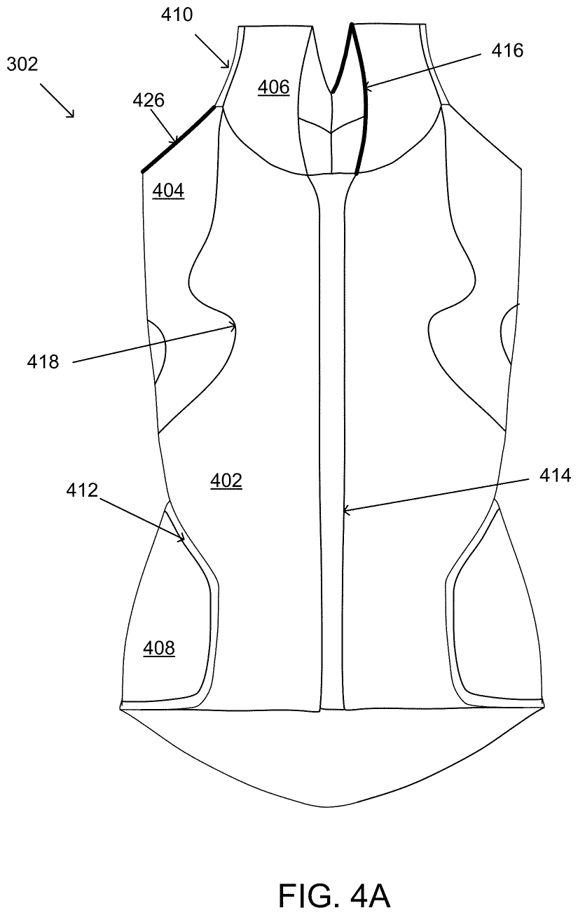

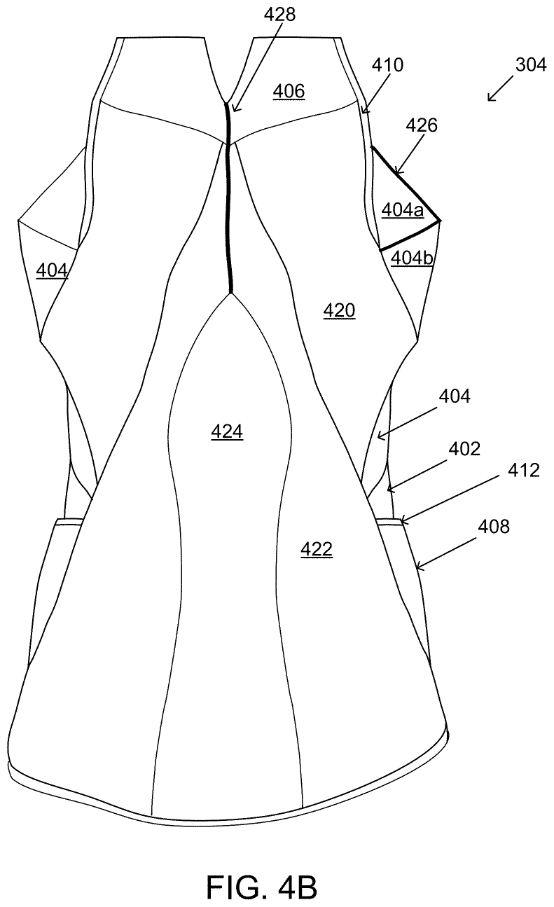

FIGS. 3A-3B and 4A-4B illustrate an exemplary garment according to embodiments described herein. The illustrations of FIGS. 3A-3B represent and exemplary combination of neurobands and posture mount panels in which the neurobands overlay exemplary muscle groups such as the trapezius, rhomboid, latissimus dorsi, and gluteus maximus. The exemplary seams between panels, bands, and combinations thereof include approximations of muscle attachment locations and/or in natural body anchor positions such as the bones and fascia locations. FIGS. 3A-3B illustrate exemplary neurobands and posture mount panels in solid lines in which exemplary fiber directions are illustrated by line arrows, bias of elasticity and/or directions of pull are illustrated by solid arrows, regions having similar bias of elasticity are indicated by letters, and exemplary underlying muscle and/or body anatomy are illustrated by dotted lines and labeled. As seen, an exemplary garment according to embodiments described herein may include a plurality of neurobands that may be configured to be positioned over the trapezius muscle and latissimus dorsi muscle of a wearer when positioned in an in use position. A combination of neurobands may be configured in mirrored arrangement to be positioned on respective sides of the spine of the wearer. In an exemplary embodiment, a combination of neurobands may be configured to be positioned to overlay respective musculature groups of the body.

In an exemplary embodiment, a garment 300 may include a back portion 304 made up of a plurality of neurobands and posture mount portions. As illustrated, the garment may include at least three neurobands on each side of the wearer. The garment may include at least three posture mount portions on the back portion. The posture mount portions may be separate panels integrated into the garment and/or may be formed by overlapping portions, such as adjacent neurobands and/or by seams between neurobands, posture mount portions, or combinations thereof. The neurobands may be more elastic than the posture mount portions. The neurobands may include directional bias of elasticity. FIGS. 3A-3B illustrates the exemplary garment in which exemplary bias of elasticity and different panel portions are illustrated. The bias of elasticity is indicated by the bidirectional arrow. The terminal ends of the respective panels are indicated in lines and may indicate a seam. In an exemplary embodiment, a seam may include overlapping panels that are bonded together, sewn, adhered, or otherwise attached.

As illustrated, exemplary neurobands may include a bidirectional bias of elasticity. The bidirectional bias of elasticity may be generally outwardly away from the spine of the wearer, and/or upward over the top of the garment. The bidirectional bias of elasticity may be oblique to the spine or vertical axis of symmetry of the garment and/or the wearer. In an exemplary embodiment, the bias of elasticity is oblique or angled relative to the vertical orientation (along the spine) of the garment. The bidirectional bias of elasticity may be perpendicular or may be oblique to each other.

As illustrated in FIGS. 3B and 4B, an exemplary garment may include a back portion 304 configured to be worn to cover a back of a wearer in an in use position. The back portion may include a plurality of neurobands and a plurality of posture mount portions. In an exemplary embodiment, the posture mount portions may be created through panels of reduced elasticity and/or may be created through the attachment between panels, such as between neurobands or between neurobands and posture mount portions, and/or through the use a seams.

In an exemplary embodiment, anchor points are created along the spine of a wearer. The anchor points are achieved through positioning of a posture mount panel and/or seams along the spine of a wearer. As illustrated in FIGS. 3B and 4B, and exemplary garment may include a posture mount panel 424 that starts at approximately the middle of a back of a wearer. In an in use or as worn position, the top of the posture mount panel 424 is positioned at or proximate to the lower rhomboid muscle group at the top of the thoraco-lumbar fascia. The posture mount panel 424 tapers outwardly from a point at the spine positioned in the middle of the garment proximate or below a position between the arm apertures. The posture mount panel 424 then tapers outward to a greater thickness as the panel traverses downward along the spine of a wearer or toward the bottom hem of the garment. The posture mount panel 424 is shaped to approximate the thoraco-lumbar fascia and run adjacent or along a lower edge of the latissimus dorsi of a wearer in an in use position. The posture mount panel 424 may then narrow, remain approximately constant or continue to taper outward toward the lower edge of the garment. As illustrated, the posture mount panel 424 narrows at the lower back of a wearer following or approximating the shape of the thoraco-lumbar fascia at the edge of the gluteus maximus.

In an exemplary embodiment, anchor points are created along the rest of the spine of the wearer. The anchor points extending above posture mount panel 424 toward a top of the garment may be defined or created by seams of neurobands, posture mount panels, and combinations thereof. As illustrated, a posture mount panel is created by seam 428 extending from a top of the posture mount panel 424 along the central region of the garment toward the opening for a head and neck of a user in an in use position.

In an exemplary embodiment, a neuroband 406 is configured to position over the top of the garment and extend over the top should of a wearer in an in use position, approximating the position of the trapezius muscle group. The neuroband 406 is configured in combination with the posture mounts, such as posture mount 428 and 424 to actively influence the body's own resources to preserve and maintain optimal alignment using biofeedback to enhance senses of proprioception, kinesthesia, and touch. In an exemplary embodiment, the neuroband 406 is configured to impose a tensile function on the trapezius muscle group directed upward from the anterior portion of the garment 302 over the shoulder toward the anterior or back of the garment 304. The neuoband 406 may have terminal ends at posture mount locations corresponding to fascia of the underlying body anatomy. For example, the approximate posture mount of the neuroband 406 may correspond on the front of the garment at, over, or proximate to the collar bone, and may correspond on the back of the garment at, over, or proximate the fascia between the trapezius and deltoid muscle proximate the shoulder blade when the garment is in an worn position. The posture mounts of neuroband 406 are positioned approximately a quarter to a half of an arm aperture from the top of the garment toward the bottom of the garment. The posture mount of neuroband 406 on the front of the garment may be positioned lower toward the bottom hem of the garment than the posture mount of neuroband 406 on the back of the garment. An exemplary representation of the relative locations of the posture mounts of neuroband 406 can be seen in FIG. 3A.

In an exemplary embodiment, a neuroband 404 extends from the portion of the arm aperture, under the arm pit of a wearer in an in use position, toward the back of the garment. The neurob and 404 is configured to impose a pulling force on the garment to counter the pulling force of neuroband 406 and reduce the pull of the garment into the armpit of the wearer. As illustrated, the exemplary garment is configured to redirect the upward force created by neuroband 404 downward and around the armpit around the body of the wearer. Neuroband 404 interacts with posture mount 418 on the front of the garment. The lower edge of the posture mount at the lower edge of neuroband 404 on the front of the garment 302 approximates the lower rib(s) of the wearer in an in use configuration. The posture mount may be angled upward from the side of the garment traveling toward the center and top of the garment approximately the lower rib cage of a wearer. Posture mount 418 at the seam or edge of neuroband 404 on the front of the garment 302 extends inward toward the sternum of a wearer in an in use configuration or center of the garment. The posture mount or edge of neuroband 404 may thereafter extend generally upwardly toward the arm aperture and/or upper portion of the garment. The edge of the neuroband 404 may intercept the arm aperture and/or neuroband 406. The neuroband 404 may extend outwardly, away from the center of the garment as it extends upwardly to position the edge of the neroband 404 and posture mount on the outside region of the chest toward the arm apertures. The location of the neurband away from the central chest and moving the posture mount to an outside location of the nipples or soft tissue of the chest of the wearer may improve comfort and reduce compression of sensitive soft tissue. The neuroband therefore may create a projection toward the center of the garment at the lower portion of the neuroband on the front of the garment over a low rib cage of the garment in an in use position, under the soft tissue of the breast of the wearer.

As illustrated in FIG. 4A, neurobands 406 and 404 may include portions that define terminal edges of the garment and/or apertures for the body of the wearer. These apertures may be positioned against soft and/or sensitive tissue of the wearer, such as the armpit and neck. Creating pulling forces along these surfaces may therefore cause discomfort to the user. Exemplary embodiments may include features to direct pulling forces around these apertures and/or configure the aperture and/or garment to reduce discomfort at these locations. For example, seams 426 and 416 of neurobands 404 and 406, respectively, may be terminal ends of a neuroband but may retain elasticity of the neuroband by not including standard sewn, bonded, adhered, or otherwise restrictive or limiting components. In an exemplary embodiment, neurobands 404 and 406 are created by a single, integrated monolithic, piece of material folded over onto itself to create seams 426 or 416. The terminal end of the garment and/or neuroband therefore does not correspond to a terminal end or edge of the material creating the neuroband. As illustrated in FIG. 4B, the neuroband 404 may include a fold over portion along seam 426 such that the interior layer 404a is created on an inside of the garment under an exterior layer 404b. For stability and/or in areas that are less prone to discomfort, conventional seams, such as from bonding, sewing, etc. may be incorporated into the garment. As illustrated in FIG. 4A, the arm aperture includes a folded seam 426 along the lower portion of the aperture and a sewn or bonded seam 410 along the top of the aperture. The top of the aperture along seam 410 may define or create another posture mount for the garment. The dual layer of the neurobands 406 and 404 may impact the elasticity of the panels. Their corresponding elasticity characteristics are indicated in FIG. 3A by letter “B”. Other areas of the garment may include single layers. As illustrated with respect to embodiments including sleeves, the neuroband 404 may be a single layer and may circumscribe the arm aperture of FIGS. 4A-4B as described herein creating either a short, partial, or long sleeve configuration.

The front of the garment 302 may include a panel 402 configured to accommodate the relative size of a user. For example, as seen in FIG. 3A, the panel 402 on the front of the garment may permit lateral expansion of the wearer and accommodate the expansion of the stomach of the wearer. As illustrated, the front panel 402 covers the chest of the wearer and the abdominals or stomach of the wearer. In an exemplary embodiment, the front panel 402 may include a posture mount, vertically from the top to the bottom of the garment in the center of the garment. The posture mount 414 may include a seam, or other reduced elasticity component. In an exemplary embodiment, the posture mount 414 comprises a zipper along part or all of the length of the garment. The zipper may permit easier positioning of the garment for use by a wearer. In an exemplary embodiment, the zipper may extend from the top of the garment toward a lower portion of the garment to approximately or at least the sternum of the wearer. The zipper may overlay the sternum to create an anchor point. In the embodiment in which the zipper extends the entire length of the garment, bi direction zipper may be included. This may permit a wearer to couple or decouple the zipper from either end of the garment. In this case, the wearer may have the zipper extended and closed along an entire length of the garment. The garment may also be configured to permit the unzipping of the garment from either of the top and/or the bottom of the garment. This may facilitate expansion or reduced compression caused by the garment, such as when seated or after eating. In an exemplary embodiment, the panel 402 may include a high elasticity as compared to other neurobands and/or posture mounts.

The back of the garment 304 may also include panels 422 to permit expansion of the garment about the back of the wearer. The panel 422 may extend between the posture mount 424 over the spine toward the lateral side of the wearer. The panel 422 may extend from the posture mount 428 and/or 424. The panel 422 may be narrower at an top end of the garment about the rib cage at the arm and arm aperture level of the garment. The panel 422 may therefore provide limited expansion of the garment along the upper back of the garment to maintain the pulling effects of the neurobands. The panel 422 may taper and increase in width as the panel 422 extends toward the bottom of the garment. This may permit greater expansion of the garment around the waist and midsection of the wearer to accommodate different physical anatomical structures of different users. The panel 422 may be mirrored on both sides of the garment about the central, vertical axis of the garment. The panel 422 may be of the same elastomeric material as the neuroband or may be of another material. In an exemplary embodiment, the panel 422, identified as panels A on FIGS. 3A-3B are of a greater elasticity than neurobands 406 or 404 or regions indicated by areas B. In an exemplary embodiment, the panel 402 and 422 that permit expansion of the garment may contact creating a continual band of elasticity from the back of the garment around the lateral side and to the front of the garment about a midsection of the garment corresponding to the stomach or belly button height of the wearer in an in use position. The maximum expansion and variation of sizes and/or in dynamic sizes of the wearer may be accommodated.

The back of the garment 304 may include posture mounts about the back rib cage of the wearer that may extend laterally around a side of the wearer. For example, the garment may include posture mount 420. The posture mount 420 may provide an anchor point for the neurobands 406 and/or neuroband 404. The posture mount 420 may cover a portion of the wearer's rib cage approximating the latissimus dorsi of the user. The posture mount 420 may also extend around a lateral side of the wearer for stability. The posture mount 420 may extend between panel 422 neuroband 406 and 404 and/or posture mount 428.

In an exemplary embodiment, additional posture mounts may be added to the garment. For example, posture mount 408 may be added about the lateral sides of the garment corresponding to the lower portion at the hem of the garment. The posture mount 408 may be configured to position over or approximate the hip area of the wearer. The posture mount 408 may be created by adding another layer over panel 402 and/or from another stand along, integrated panel into the garment. In an exemplary embodiment, a second layer is added from the panel 422 overlaying a portion of panel 402. The second layer 408 may be coupled to the underlying layers by a seam 412. The seam and/or combination of the second layer may reduce the elasticity of this area and create an anchor point to the body. In an exemplary embodiment, the first and second layer may move independent of each other and may be unattached along a portion of the layer. For example, the top edge of layer 408 may be unattached to the underlying garment to permit separation of the garment at this location. In an exemplary embodiment, the second layer 408 may be used to create a pocket or holder having an opening at a top of the pocket. In an exemplary embodiment, the second layer 408 may be removably attached to the underlying layer 402. In this way, the relative size of the garment may be configurable, such as by relocating the attachment point of the forward terminal end of the panel 408 to the panel 402. The removable attachment may be through hook and loop fastener, hook/eye, snaps, buttons, or other desired coupling.

In an exemplary embodiment, seams may be created by a bonding of the material, and/or in the addition of an elastic band sewn to a panel, or other method used at an edge of a garment and/or to couple panels of a garment together. As illustrated, seams 410 and 408 include an elastic band sewn and/or bonded to the panel.

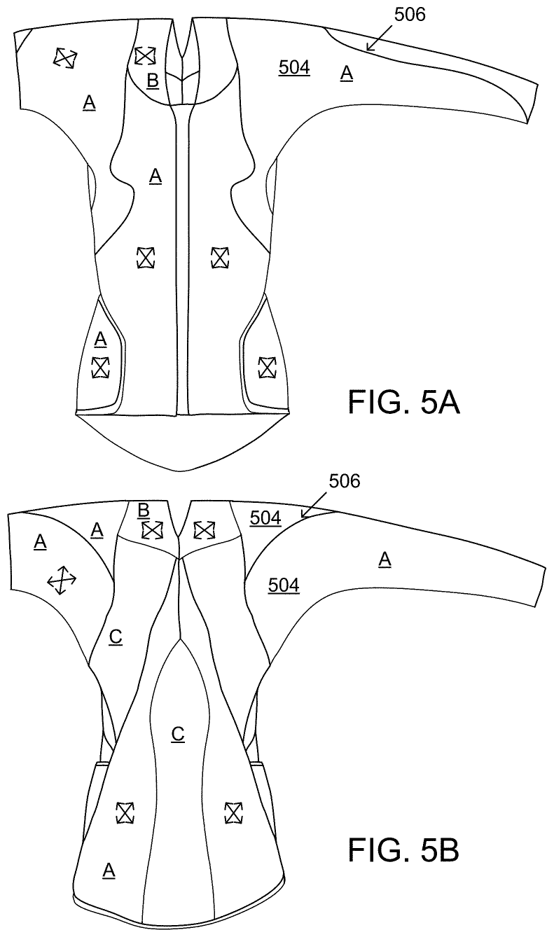

FIGS. 5A-5B illustrate exemplary embodiments of a garment including sleeves. For sake of illustration only, various sleeve configurations are illustrated on opposite sides of the garment. Garments according to embodiments described herein may include the same sleeve configuration and/or length on both sides of the garment. Exemplary embodiments of a sleeved garment may include a sleeve of any length. A short sleeve and long sleeve are illustrated for sake of example, but other sleeve lengths, such as ½ or ¾ sleeves are also within the scope of the present description.

As illustrated in FIG. 5A, the exemplary garment includes a neuroband 405 similar to neuroband 404 of FIGS. 4A-4B. The neuroband 405 extends from posture mount and circumferentially wraps around the previous arm aperture of FIGS. 4A-4B created by posture mount 420 and neuroband 406. Therefore, instead of seam 410 and 426, the neuroband 504 continues around the aperture to contact itself along seam 506. The configuration of the extension of neuroband 504 repositions any seam away from the arm pit of the wearer to reduce pulling of the garment into the armpit. In an exemplary embodiment, seam 506 starts on the back of the garment about the middle of previous arm aperture. The position of the seam starts over or proximate a shoulder blade or other anchor point of the wearer. The seam then extends outward and upward to spiral over the top of the garment and along the length of the sleeve. In an exemplary embodiment, a portion of the seam 406 follows the deltoid fascia of the user in an as worn position. The seam 406 then extends along the length of the sleeve along the triceps extending along the forearm. Adjacent a wrist of the wearer or terminal end of a long sleeve version of the garment, the seam 406 may spiral around about a quarter of the sleeve circumference and extend across the wrist of the wearer from adjacent the brachio radialis across the radius toward the flexor retinaculum. The seam therefore traverses across the sleeve from a top region of the sleeve (thumb side of a wearer's hand) to a lower region of the sleeve (pinky side of a wearer's hand). The sleeve may provide additional pulling of the garment out of the armpit and influence the orientation of the arm and/or shoulder posture of the wearer in an in use position. Therefore, the sleeve from neuroband 504 may be a single layer as opposed to the dual layer of FIGS. 4A-4B. The neuroband 504 may therefore have a greater elasticity than the corresponding neuroband 406. As illustrated, areas indicated with a letter A may have a greater elasticity or amount of stretch than compared to panels identified by letter B; both of which A and B may have greater elasticity or amount of stretch than compared to panels identified by letter C.

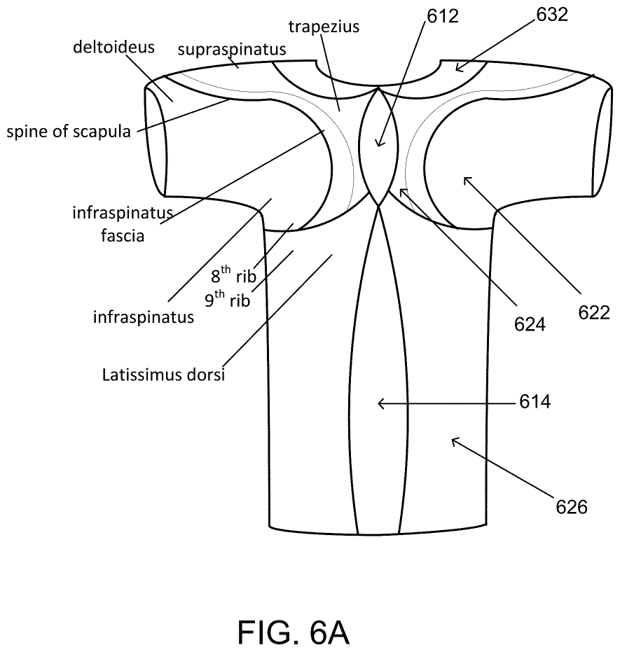

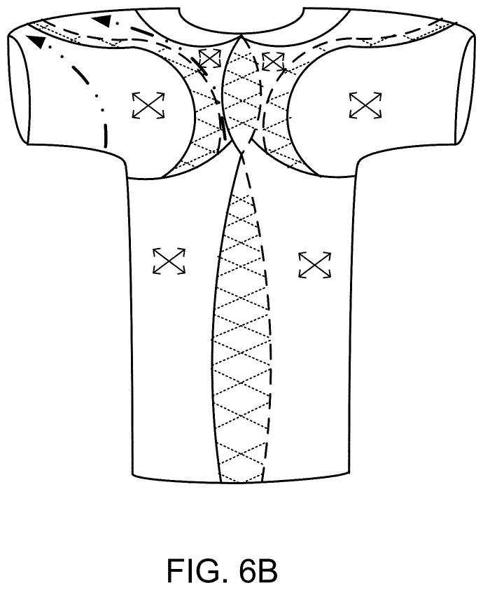

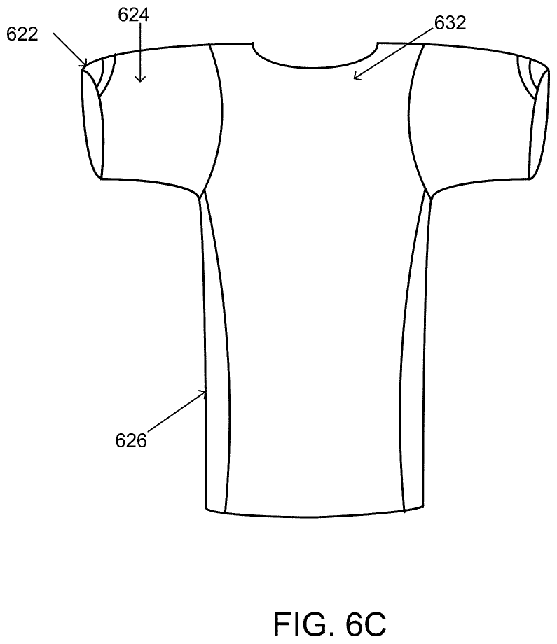

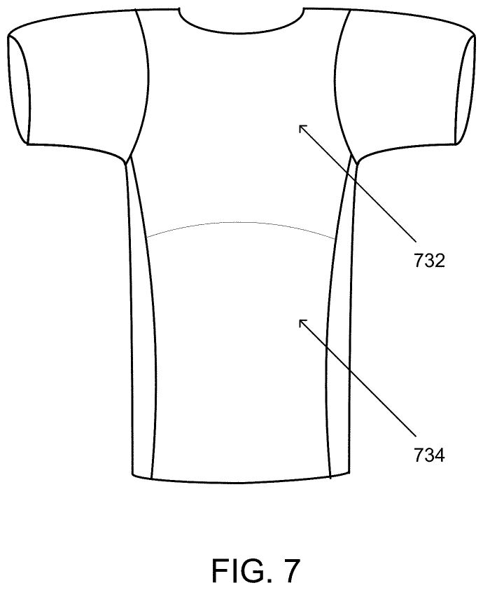

FIGS. 6A-6C illustrate an exemplary garment according to embodiments described herein. FIG. 6A illustrates a back portion of the garment having a plurality of neurobands and posture mount portions. A neuroband may be configured to be positioned over the trapezius muscle and latissimus dorsi muscle of a wearer when positioned in an in use position. A combination of neurobands may be configured in mirrored arrangement to be positioned on respective sides of the spine of the wearer. In an exemplary embodiment, a combination of neurobands may be configured to be positioned to overlay respective musculature groups of the body. For example, as illustrated in FIG. 6A, a first neuroband 624 may position adjacent and/or overlay the spine of the wearer and extend radially upward and outward toward and the shoulder of the wearer. The exemplary first neuroband 624 is configured to position and overlay the trapezius. The exemplary first neuroband 624 may extend at approximately near or over the lower trapezius and extend upward along a spine and outward along the supraspinatus muscle of the wearer. A second neuroband 626 may be positioned adjacent the spine of a wearer and be configured to overlay the latissimus dorsi muscle of the wearer in an in use position. A third neuroband 622 may be positioned over the teres minor, teres major, and infraspinatus muscle areas.

In an exemplary embodiment, a garment may include a back portion made up of a plurality of neurobands and posture mount portions. As illustrated, the garment may include at least three neurobands on each side of the wearer. As illustrated, the garment may include at least three posture mount portions. The posture mount portions may be separate panels integrated into the garment and/or may be formed by overlapping portions, such as adjacent neurobands. The neurobands may be more elastic than the posture mount portions. The neurobands may also include directional bias of elasticity. FIG. 6B illustrates the exemplary garment of FIG. 6A in which exemplary bias of elasticity and overlapping panel portions are illustrated. The bias of elasticity is indicated by the bidirectional arrow. The overlapping portions of adjacent panels are indicated by the dotted cross-hatched lines, and the terminal ends of the respective panels are indicated in lines, in which the exterior terminal end of a panel is solid and an interior terminal end of a panel is dashed. For example, the first neuroband overlaying the trapezius overlays or is exterior to the third neuroband overlaying the infraspinatus. However, the reverse configuration is also encompassed by the present disclosure.

FIGS. 6A-6B illustrate an exemplary embodiment of a garment according to embodiments described herein. An exemplary first pair of neurobands may be positioned on opposing sides of the back of the garment at a lower portion of the garment. The first pair of neurobands may overlap in a central region of the garment overlaying a spine of a wearer in an in use position. The overlapping neurobands may create a first posture mount portion. The first pair of neurobands may extend generally below the shoulder blade area around the eighth or ninth rib of a wearer proximate and/or below the infraspinatus muscle area of the wearer in an in use position. As illustrated the first pair of neurobands extend from below the armpit area of the garment to a lower or bottom end of the garment. In an exemplary embodiment, the first posture mount portion defines an ovoid area. A second pair of neurobands are vertically above the first pair of neurobands and overlap over a central area of the garment covering the upper spine of the wearer when in an in use position. The overlapping area of the second pair of neurobands may define a second posture mount. The second posture mount may define an avoid area. The second pair of neurobands extend from the first pair of neurobands up and over a shoulder and/or arm of the wearer. An ovoid shape is generally curved in which opposing ends of the shape may be tapered. Ovoid shapes may also include circular shapes.

In an exemplary embodiment, each of the respective second pair of neurobands extend from a corresponding each of the first pair of neurobands and extend up and over the top of the garment. Each of the respective second pair of neurobands may then circumscribe, or wrap around an arm aperture of the garment and reattach to either the respective one of the first pair of neurobands and/or to itself on the back side of the garment. For example, a single panel may create and define the arm aperture of the garment. As illustrated in FIG. 6B, the double dot-dashed arrow shows the projection of a single, unified, non-segmented, panel extending from one of the first pair of neurobands upward over a shoulder portion of the garment, wrapping around the arm aperture, and around to define and create the sleeve.

In an exemplary embodiment, the garment sleeve is made of a single panel having only a single, continuous seam coupling the sleeve to the garment and to itself. The exemplary garment defines a curved seam that extends along and proximate the shoulder blade of the wearer in a worn position. For example, a portion of the seam may overlay the bone of the shoulder blade, the infascpinatus fascia, and outward along the spine of scapua. The seam may define a pasture mount and may overlay anatomical attachment areas of the muscle groups to the body, such as over fascia or bone. In an exemplary embodiment, the seam created between one of the second pair of neurobands and itself is a non-woven seam. The seam may be created by overlapping the neuroband and adhering the neuroband to itself. The adhesion may be through an infusion of an elastic material into the material of the neuroband. Exemplary embodiments include an elastic seam. The elastic seam may be creating through impregnating an elastic into the material, by adhering elastic materials, by using elastic materials, and combinations thereof.

In an exemplary embodiment, garment may include expansion panel 632. The expansion panel may extend around a substantial majority of the neck aperture around the back of the wearer, and around the front of the garment. The expansion panel 632 may form the front of the garment. The expansion panel have a reduced bias of elasticity as compared to the bias of elasticity of the neurobands. FIG. 6C illustrates an exemplary front portion of a garment as illustrated in FIGS. 6A and 6B.

Exemplary embodiments described herein may include exemplary neuroband with markings and/or apertures. The markings or apertures may be used to indicate an amount of stretch of the material at or along the panel. For example, the spacing between markings, and/or the elongation of the shape of an aperture may indicate an amount and/or direction of pull on the garment. This may be useful, especially when the garment is used with additional taping or imposed forces. The apertures may also or alternatively be used for breathability, cooling, wicking, or other purpose or benefit.

FIG. 7 illustrates an exemplary front of a garment including features that may be incorporated into any configuration described herein. The exemplary embodiment may include a plurality of panels 732, 734. The panels may have the same or different attributes. As illustrated a second expansion panel 734 is positioned on a lower portion of the front of the garment that would overlay a stomach area of a wearer in a worn position. The second expansion panel 734 may be made of a substantially higher elasticity and in one or more directions to assist in the expansion and accommodation of different sizes of the anatomical region across different users. The first expansion area 732 may be used as described herein to support and permit general movement of the wearer, but provide reduced elasticity to provide support for the rest of the garment and the neurobands as described herein. In an exemplary embodiment, a transition to or top of the second expansion panel 734 corresponds proximate to or at the lower rib cage of the wearer.

Exemplary embodiments include a system of using exemplary embodiments described herein. Exemplary embodiments may use, place, or adjust the location and tension of neurobands to create a form fitting posture garment corresponding to a specific user. Exemplary embodiments may include positioning a posture garment according to embodiments described herein on a portion of a body part of a user, such as the torso, arm, leg, etc. For positioning of a shirt garment over the torso of a user, the garment may be positioned over the hips of the user. Exemplary embodiments include positioning neurobands according to embodiments described herein onto the garment using the posture mount locations. Exemplary embodiments include selecting neurobands according to their bias of stretch and tensile strength to correspond to an associated muscle group for which the neuroband will overlay once positioned at the posture mounts. Exemplary embodiments include positioning the neurobands between at least two posture mounts to position and orient the neuroband to support, facilitate, correspond to, or combinations thereof to the underlaying muscle group to which the neuroband is positioned thereover.

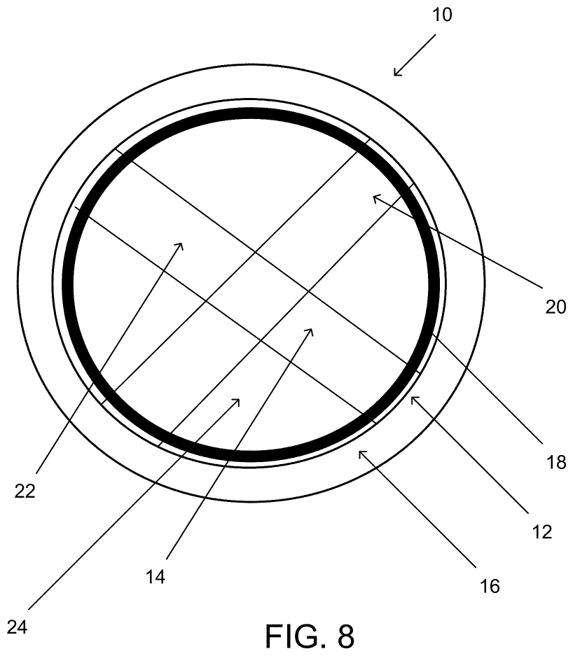

FIG. 8 illustrates an exemplary modular system for neurobands and applications thereof according to embodiments described herein. In an exemplary embodiment, modular system 10 according to an exemplary embodiment includes a perimeter 12 and an interior portion 14. In the illustrated embodiment, the perimeter 12 is circular. Other perimeter shapes may be provided instead, including oval, rectangular or other shapes. The perimeter 12 of certain embodiments is closed, forming a continuous perimeter, although modular systems having an open perimeter may also be provided. The perimeter 12 may include neuroband 16 such as fabric tape, single, or multiple biased elasticity strip, or other configuration, that may be sewn or otherwise attached to a garment. The neuroband 16 may be of woven material or non-woven material. The neuroband may be of an extruded or otherwise formed material. The neuroband 16 may be affixed to a perimeter by thread, by adhesive, by welding or other affixing means, or by a combination of affixing means, such as those described herein. The neuroband 16 of certain embodiments is flexible but may be generally resistant to stretching. In other embodiments, the tape 16 is of a stretchable material.

The perimeter 12 of certain embodiments may also include a border 18. The border 18 of certain embodiments may include cording or other material within the border 18 and may be wrapped in a fabric or other cover. The border 18 may instead include a unitary member. For example, an extruded material may be provided as the border 18. In certain embodiments, the border 18 is resistant to stretching. In other embodiments, the border is of stretchable material.

The border 18 of certain embodiments encloses the interior portion 14. In the illustrated example, the interior includes two portions 20 and 22 that are connected at their ends to the border 18. In particular, the portion 20 extends along a diameter of the circular biomechanical component 10 in one direction and the portion 22 extends along a diameter of the circular biomechanical component 10 in another direction. The two portions 20 and 22 cross generally at a center of the component 10. The portions 20 and 22 may be formed of elastic material, such as elastic fabric straps, that apply tension along their lengths. The straps 20 and 22 may be positioned at right angles to one another, as shown, or at other angles as desired. The primary direction of force for the component 10 may be defined by the straps 20 and 22.

The interior portion 14 may also include an area 24 that is not covered by the straps 20 and 22. The area 24 may be left open or may include a fabric, such as a stretch fabric, which covers the area not covered by the straps 20 and 22. For example, a thin, stretch fabric may be provided within the border 18, either under or over the straps 20 and 24.

The biomechanical component 10 may be provided in a garment or other article of clothing. In certain embodiments, the biomechanical component 10 is provided in tight fitting clothing or in a tight fitting portion of an article of clothing. The active tension provided by the perimeter 12 and interior portion 14 may have the most effect in tight fitting portions of clothing. Exemplary embodiments of the biomechanical component 10 may be attached, either permanently or removably, to an underlying garment. Permanent attachment may include a semi-permanent attachment that may require additional intervention to remove, such as adhesion, sewing, bonding, etc. Removable attachment may be any attachment that a user and add or remove during the normal course of use, that may not require application or use of outside components or products. For example removable attachment may be through hook and loop, buttons, snaps, etc. Exemplary embodiments of the biomechanical component 10 may be to provide additional neurobands to a garment in specific and configurable locations to an individual user.

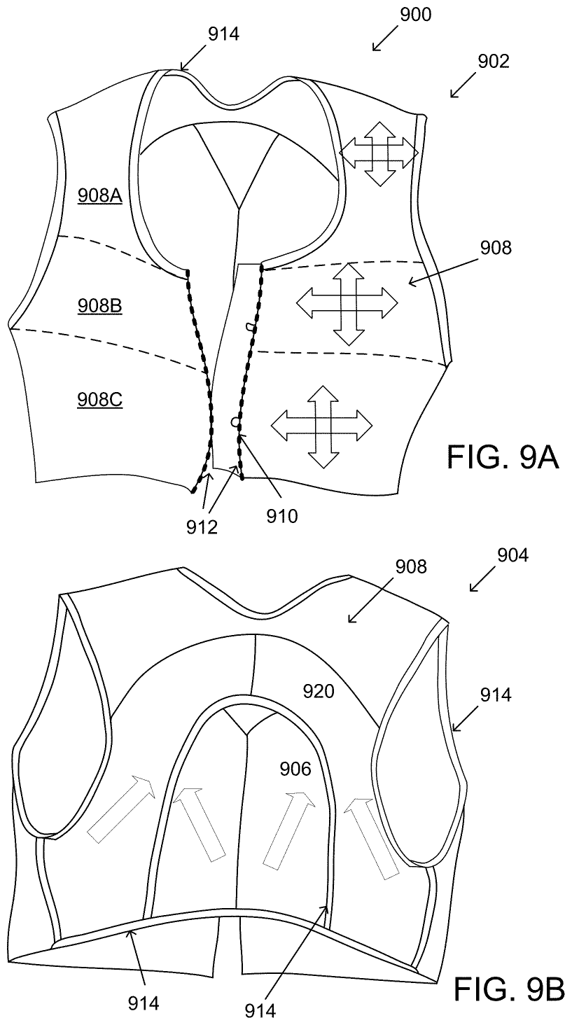

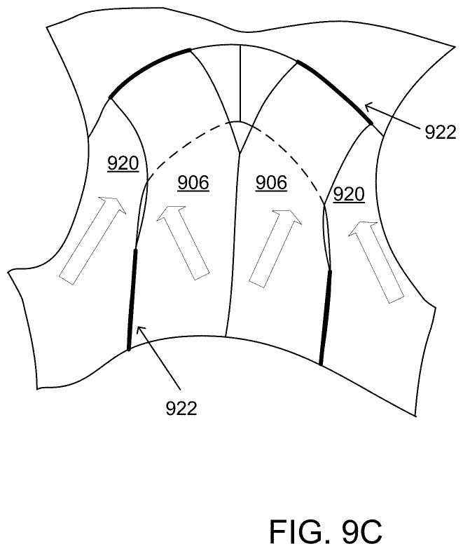

FIGS. 9A-9C illustrates an exemplary neuroband configuration for use as a half top. The exemplary garment 900 includes a back portion 904 and a front portion 904 as illustrated in FIGS. 9B and 9A, respectively. FIG. 9C illustrates an exemplary view of the interior side of the back side of the garment, corresponding approximately to FIG. 9A with the front portions of the garment including 908A, 908B, 908C removed from view for a better understanding of the interaction of neurobands 906 and 920.

In an exemplary embodiment, the front of the garment 902 may include a single panel that extends from the bottom of the garment, over the chest of a wearer in an in use position, over the shoulder of the wearer or over the top of the garment, traversing from the front to the back of the garment, across the back of the garment, traversing around the back of the neck or upper shoulder/spine of the wearer in an in use configuration, and over the should of the wearer or over the top of the garment, and back down the other front side of the garment, over the chest, and to the bottom of the garment. The front panel 908 may be elastic to accommodate the variability of chest sizes of individual users. In an exemplary embodiment, the front panel 908 may include one or more sections to permit different elasticity within regions of the garment. The sections may be created through various fabric patterns, reduced density fabric areas, reinforcement layers, or in separate panels attached together. In an exemplary embodiment, the front panel 908 is made of a single, unitary panel that incorporates different fabric orientations, densities, patterns, weaves, etc. to create different elasticities in the various sections. For example, the front panel 908 may include two, three, or more sections.

In an exemplary embodiment, a section 908C may correspond to a lower or bottom region of the garment. The first section 908C may have a reduced elasticity compared to one or more other sections to stabilize and create support for the user's soft tissue of the chest and for supporting the rest of the garment including the neurobands described herein. In an exemplary embodiment, the first second 908C may be created by multiple layers of material. For example, two layers may be separately attached and/or a single material panel may be folded over. In an exemplary embodiment, a bottom edge of the garment is created by a fold over of the material that is sewn, bonded, or otherwise attached to the garment to define a lower band.

In an exemplary embodiment, a section 908B may corresponding to an interior portion of the front panel 908 that generally covers the soft tissue of a wearer's breast in an in use position. The second section 908B may have a greater elasticity as compared to one, more, or all of the panels of the garment. The increased elasticity may be created by reducing the material panel layers as compared to, for example, section 908C, or by reducing the fiber density, fiber pattern, or combinations thereof.

In an exemplary embodiment, the front panel 908 may include a section 908A that corresponds to an upper portion of the front panel 908. The top section 908A may be an extension of the section 908B with the same elasticity or may have a different elasticity from the interior portion of section 908B. The top section 908A may extend over the top of the garment and extend from the front of the garment to the back of the garment. The top section 908A may extend around and across the back of the garment. The top section 908A may define and/or be adjacent to the neck aperture and/or at least a portion of the arm aperture of the garment. The top section 908A may have an elasticity between that of the bottom section 908C and the central section 908B. The top section 908A may have an elasticity that is approximately the same as the bottom section 908C or as the central section 908C.

In an exemplary embodiment, the front panel 908 may be separable, and/or attachable. The front panel 908 may include a zipper 912, hook/eye fastener 910, or there combination of connections, such as, for example, buttons, hooks, snaps, hook and loop, etc. In an exemplary embodiment, the garment includes a neck aperture and two arm apertures. In an exemplary embodiment, the apertures include an elastic portion 914 to replace a hem. The hem according to embodiments described herein may include a separate elastic piece folded around the edge of one or more panels to create an edge of the garment. The hem may also include a material bonded or impregnated into the fabric to retain the fabric while permitting elastic flexibility.

FIG. 9B illustrates an exemplary back 904 of the garment according to embodiments described herein including neurobands. As illustrated, the back of the garment includes a plurality of neurobands 906, 920. In an exemplary embodiment, a first pair of neurobands 920 create a boarder or frame for which the second pair of neurobands 906 are positioned. The boarder may be similar to the perimeter 12, neuroband 16, and/or boarder 18 of FIG. 8. The pair of neurobands 920 define a arc, or ovoid opening at the back of the garment. The opening may have a hem 914 as described herein. The pair of neurobands 920 may comprise a directional elasticity. As illustrated by solid arrows, the bias of elasticity of the neuroband 920 is oblique to the vertical axis of the garment that would be positioned along a spine of a wearer in an in use position. The pair of neurobands 920 may be adjacent or define a portion of the arm aperture. The pair of neurobands 920 may couple on the later sides of the garment to the front panel 908 and at the back of the garment along the top of the neurobands 920 to the front panel 908. The top edge traversing the pair of neurobands 920 defines an arc across the back of the wearer in an in use position. The top edge of the neuroband 920 starts in a middle region of the arm aperture and extends upward toward the neck aperture as the top edge is traversed across the garment. In an exemplary embodiment, the top edge approximates the location of the edge of the deltoid, and/or infraspinatus and traverses the trapezius.

In an exemplary embodiment, the garment includes a pair of neurobands 906. The pair of neurobands is configured to be positioned over the trapezius muscle group have a bias of elasticity in an approximately direction of the muscle fibers of the trapezius. As illustrated, the pair of neurobands 906 extend across the aperture created by the pair of neurobands 920 along the bottom of the garment. The pair of neurobands 906 extend upward and are coupled directly together vertically along a vertical center of the garment, configured to be positioned over a spine of a wearer in an in use position. The attachment between neurbands 906 may define an posture mount. The pair of neurbands 906 are unattached along a length of the bands toward the top or toward the neck aperture of the garment. The pair of neurobands are angled away from the vertical axis of the garment corresponding to a spine of a user in an in use configuration and away from each other. The pair of neurobands 906 extend away from each other away from the vertical axis of the garment and up toward the shoulder of the garment where the front panel 908 extends up and over the top of the garment. The pair of neurobands 906 may extend under or over and overlap with the pair of neurobands 902 along a portion of the respective neurobands along a top region of the neurobands. In an exemplary embodiment, the pair of neurobands 906 couple to the front panel 908 at a top portion of the neuroband 906 on the back of the garment 904.

The relationship of an exemplary first pair of neurobands 920 relative to a second pair of neurobands 906 is illustrated by the cut away view of FIG. 9C illustrating an interior side of the back portion of the garment. A first one of a first pair of neurobands 920 extends from a lateral side of the garment and a bottom of the garment upward toward the shoulder of the garment and arcs toward the neck aperture of the garment and toward the central vertical axis of the garment over a spine of the wearer in an in use configuration. A second done of the first pair of neurobands 920 is in mirrored relationship from the first one of the first pair of neurobands 920. The first pair of neurobands creates an arc configuration from the bottom edge of the garment with a separation between the first one and the second one of the first pair of neurobands 920. The second pair of neurobands 906 are positioned within the separation created by the first pair of neurobands 920. A first one of the second pair of neurobands is coupled to the first one of the first pair of neurobands along a length 922 along a lower portion of the respective neurobands. The first one of the second pair of neurobands 906 is coupled along a second length to the second one of the second pair of neurobands 906 along approximately a center, vertical orientation of the garment. The second length of the attachment of the second pair of neurobands 906 to each other may be longer than the attachment length 922 between one of the second pair of neurobands to one of the first pair of neurobands. The second pair of neurobands 906 diverge upwardly and away from each other after the end of the attachment length. The second pair of neurobands 906 are illustrated as extending on an interior side of the first pair of neurobands 920 to create a second layer with the first pair of neurobands. The dotted line indicates the continuation of the first pair of neurobands behind the second pair of neurobands. The second pair of neurobands 906 may traverse a width of the first pair of neurobands to couple to the garment and to the first pair of neurobands at a top or exterior edge of the first pair of neurobands away from the separation space. An exemplary connection location 922 is at a terminal end of the neuroband 906 toward a shoulder of the garment. The first pair of neurobands 920 and second pair of neurobands 906 may be unattached along a length traversing any of the neurobands of the first pair of neurobands. The each of the second pair of neurobands 906 may therefore move locally relative to each of the first pair of neurobands across the width of either of the first pair of neurobands. The first pair of neurobands and second pair of neurobands are statically positioned with respect to one another only along an exterior edge of the neurobands.

Exemplary embodiments of neurobands are described herein. Exemplary embodiments of garment may include any combination of features as described herein. For example, an opening of one embodiment may be combined with one or more neuroband arrangement of another embodiment. The neurobands may be combined in any combination as would be understood by a person of skill in the art and the selected configurations are provided for illustration purposes only.

Exemplary embodiments described herein include a component including a neuroband. The component may be integrated or define a garment, a sleeve, or a modular attachment. The garment according to embodiments described herein may also include any combination of a posture mount, an expansion panel, and/or a neuroband, or any combination of one or more of any of these. In an exemplary embodiment, the neuroband comprises at least one bias of elasticity. The garment may include a plurality of neurobands where a first neuroband is configured to wrap around an arm portion of a wearer and define a sleeve, a second neuroband is positioned over a shoulder of a wearer from the front of the garment to a back of the garment. Exemplary embodiments include posture mounts over a spine of a wearer and along the shoulder blade and/or collar bone of a wearer in an in use position.

In an exemplary embodiment, a neuroband is created by overlapping a material to create a multi-layered portion of the garment. The neuroband may include a bias of elasticity and the overlapping material may be oriented such that a first layer and the second layer do not align the bias of elasticity. A fold over of the neuroband may defines a portion of an aperture of the garment, such as, for example, a neck or arm aperture.

Exemplary embodiments of a garment are described herein having a plurality of neurobands, a plurality of posture mounts, a plurality of expansion panels, and combinations thereof. An exemplary posture mount may be along a spine of a wearer when the garment is in an in use position. The posture mount may include anchor panels, seams, overlapping layers, or combinations thereof. For example, a posture mount may include an anchor panel having a material that is less elastic than other portions of the garment. The posture mount may include a seam extending between the anchor panel and a neck aperture of the garment. The anchor panel may extend from a lower terminal end of the garment upward toward a top of the garment along a center of the garment on the back portion of the garment. The anchor panel may taper near a top of the anchor panel toward the neck aperture of the garment. The garment may include a pair of neurobands extending over a top of the garment from the front portion to the back portion. The pair of neurobands may directly connect to each other on the back portion of the garment and extend on opposite sides of a neck aperture of the garment. The neurobands may comprise a more elastic material than the posture mount and/or anchor panel. The neurobands may include a bi-directional bias of elasticity. The axis of elasticity of the bi-directional bias of elasticity may be oblique to each other. One of the pair of neurobands may extend from the front of the garment around a first lateral side of the garment under a first arm aperture of the garment and a second of the pair of neurobands may extend from the front of the garment around a second lateral side of the garment under a second arm aperture of the garment. The garment may include a first pair of neurobands, a second pair of neurobands, a third pair of neurobands, or combinations thereof. The first, second, third, or other combination of neurobands may have any configuration in any combination as described herein.

In an exemplary embodiment, the pair of neurobands wrap around each of the arm apertures to form a sleeve on the garment. Each of the pair of neurobands contacts itself to circumscribe the arm aperture on the back portion of the garment. A seam along which each of the second pair of neurobands contacts itself may extend from the back portion of the garment upward toward a top of the garment and onto the front portion of the garment as the seam extends along a length of a sleeve.

In an exemplary embodiment, a pair of neurobands taper toward a center of the garment on the front portion of the garment as the second pair of neurobands extend downward toward a bottom portion of the garment. The taper may create an extension of each of the pair of neurobands that is configured to be positioned over a lower portion of a wearer's rib cage in an in use position. In an exemplary embodiment, a pair of posture mounts extend on the back portion of the garment from respective terminal ends of the first pair of neurobands. The pair of posture mounts may extend downward along the back portion of the garment from the pair of neurobands and around opposite lateral sides of the garment under arm apertures of the garment toward the front portion of the garment.

In an exemplary embodiment, the garment may include a first pair of neurobands and a second pair of neurobands, wherein the first pair of neurobands overlap the second portion of neurobands. The plurality of neurobands may include a first pair of neurobands and a second pair of neurobands, the first pair of neurobands are configured to define an arcuate shape having a separation between at least a portion of a first one of the first pair of neurobands and at least a portion of a second one of the first pair of neurobands. The second pair of neurobands may be positioned in the separation between the first pair of neurobands. The first pair and second pair of neurobands may include a bias of elasticity. An axis of the bias of elasticity of the first pair and second pair of neurobands may be perpendicular or oblique to each other. The axis of the bias of elasticity between each of the first pair or second pair of neurobands may be perpendicular or oblique to each other. For example, a first one of the first pair of the neurobands may be perpendicular or oblique to either of a second one of the first pair of the neurobands or a first one of the second pair of the neurobands where the first one of the first pair and the first one of the second pair of neurobands are on the same side of the garment and on an opposite side of the garment from the second one of the first pair and the second one of the second pair of neurobands. The a bias of elasticity of the first pair of the neurobands may go toward the center axis of the garment as the garment is traversed either upward or downward, while a bias of elasticity of the second pair of the neurobands may go away from the center axis of the garment as the garment is traversed either upward or downward such that the bias of elasticities of the first pair converge or diverge with respect to each other.

In an exemplary embodiment, a garment having a first pair of neurobands may create an ovoid or arch shape in which a separation is created along a lower edge of the neurobands, and also has a second pair of neurobands that overlap the first pair of neurobands. The first pair of neurobands may be coupled along terminal edges to terminal edges of the second pair of neurobands such that the first pair of neurobands can move relative to the second pair of neurobands along a portion of overlap.

In an exemplary embodiment, the garment may include a front panel that has sections of variable elasticity. The garment may also have a zipper on the front portion of the garment.

In an exemplary embodiment, the neuroband may be wrapped around and coupled to itself to define a sleeve. The sleeve may include a seam extending along a length, and the seam is curved along an entire length thereof when the sleeve is in a planar configuration.

Exemplary embodiments described herein may comprise materials having a systematic property of elasticity including single and/or multi-directional orientations. In an exemplary embodiment, materials are knit or woven in an oblique orientation to avoid or reduce perpendicular alignment of the fibers. Exemplary embodiments comprise a warp weave with or without a weft. Exemplary embodiments comprise woven or knit materials infused with an elastic material. Woven or knit materials may include nylon. Elastic materials may include lycra, spandex, elastomer, etc. Exemplary embodiments comprise materials having a warp weave and/or warp knit with and without a weft. In an exemplary embodiment, a weft may comprise titanium strands. An exemplary neuroband may include a dual bias of elasticity. The bi-directional direction of elasticity may permit the neuroband to expand a greater amount in one direction than in another direction under the same application of force. The bi-directional elasticity may permit the neuroband to expand the same amount or different amounts along each of the axis or bias of elasticity. For example, a neuroband may comprise a warp weave having a bi-directional axis of elasticity, where each of the axis of elasticity are oblique to each other. The first axis or bias of elasticity may be greater than the second axis or bias of elasticity such that the material stretches along the first axis by a greater amount than it would along the second axis given the same amount of force. However, the material may stretch less than either of the axis of bias or elasticity in a direction perpendicular to either of the axis of elasticity. In an exemplary embodiment, an exemplary neuroband comprises a bi-directional bias of elasticity. Each of the axis of elasticity are oblique to each other. In an exemplary embodiment, the elasticity along each axis is approximately equal. In an exemplary embodiment, the elasticity along a first axis is approximately 10%-40% more than the elasticity along the second axis.

In an exemplary embodiment, one or more and/or all of the seams of a garment may be bonded. In an exemplary embodiment, one or more and/or all of the seams of the garment may not be sewn. In an exemplary embodiment seams may be bonded by integrating a material into and between the panels of the seam.

Exemplary embodiments of a neuroband, seam, or bond described herein may include woven or knit material infused with an elastic. In an exemplary embodiment, a method of making such an infused material may include providing a fabric material such as a knit or woven material and a sheet of an elastic material. In an exemplary embodiment, the fabric and the sheet may be overlayed over each other. In an exemplary embodiment, the bias of stretch of the sheet and the fabric may be positioned to desired orientation. The bias of stretch of the sheet and the fabric may be parallel, oblique, perpendicular, or other orientation. In an exemplary embodiment, the elastic material is heated to infuse the elastic in the fabric. In an exemplary embodiment, the fabric may comprise nylon or a nylon blend. The elastic may be an elastomer, such as spandex.

Although embodiments of this invention have been fully described with reference to the accompanying drawings, it is to be noted that various changes and modifications will become apparent to those skilled in the art. Such changes and modifications are to be understood as being included within the scope of embodiments of this invention as defined by the appended claims. Specifically, exemplary components are described herein. Any combination of these components may be used in any combination. For example, any component, feature, step or part may be integrated, separated, sub-divided, removed, duplicated, added, or used in any combination and remain within the scope of the present disclosure. Embodiments are exemplary only, and provide an illustrative combination of features, but are not limited thereto.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. A garment including a front portion and a back portion, the garment comprising:
a plurality of neurobands; and
a plurality of posture mounts.

2. The garment of claim 1, wherein a first posture mount is along a spine of a wearer when the garment is in an in use position.

3. The garment of claim 2, wherein the first posture mount comprises an anchor panel comprises a material that is less elastic than other portions of the garment.

4. The garment of claim 3, wherein the first posture mount comprises a seam extending between the anchor panel and a neck aperture of the garment.

5. The garment of claim 4, wherein the anchor panel extends from a lower terminal end of the garment upward toward a top of the garment along a center of the garment on the back portion of the garment.

6. The garment of claim 5, wherein the anchor panel tapers near a top of the anchor panel toward the neck aperture of the garment.

7. The garment of claim 1, wherein a first pair of neurobands extend over a top of the garment from the front portion to the back portion.

8. The garment of claim 7, wherein the first pair of neurobands directly connect to each other on the back portion of the garment and extend on opposite sides of a neck aperture of the garment.

9. The garment of claim 1, wherein a one of a second pair of neurobands extend from the front of the garment around a first lateral side of the garment under a first arm aperture of the garment and a second of the second pair of neurobands extend from the front of the garment around a second lateral side of the garment under a second arm aperture of the garment.

10. The garment of claim 9, wherein each of the second pair of neurobands wrap around each of the arm apertures to form a sleeve on the garment.

11. The garment of claim 10, wherein each of the second pair of neurobands contacts itself to circumscribe the arm aperture on the back portion of the garment.

12. The garment of claim 11, wherein a seam along which each of the second pair of neurobands contacts itself extends upward toward a top of the garment and onto the front portion of the garment as the seam extends along the length of sleeve.

13. The garment of claim 9, wherein the second pair of neurobands taper toward a center of the garment on the front portion of the garment as the second pair of neurobands extend downward toward a bottom portion of the garment.

14. The garment of claim 13, wherein the taper creates an extension of each of the second pair of neurobands that is configured to be positioned over a lower portion of a wearer's rib cage in an in use position.

15. The garment of claim 8, further comprising a pair of posture mounts extending on the back portion of the garment from respective terminal ends of the first pair of neurobands.

16. The garment of claim 15, wherein the pair of posture mounts extend downward along the back portion of the garment from the first pair of neurobands and around opposite lateral sides of the garment under arm apertures of the garment toward the front portion of the garment.

17. The garment of claim 1, wherein the plurality of neurobands comprises a first pair of neurobands and a second pair of neurobands, wherein the first pair of neurobands overlap the second portion of neurobands.

18. The garment of claim 1, wherein the plurality of neurobands comprises a first pair of neurobands and a second pair of neurobands, the first pair of neurobands are define an arcuate shape having a separation between at least a portion of a first one of the first pair of neurobands and at least a portion of a second one of the first pair of neurobands.

19. The garment of claim 18, wherein the second pair of neurobands is positioned in the separation between the first pair of neurobands.

20. The garment of claim 19, wherein the first pair of neurobands create an ovoid or arch shape in which the separation is along a lower edge of the neurobands, and the second pair of neurobands overlap the first pair of neurobands.

21. The garment of claim 20, wherein the first pair of neurobands are coupled along terminal edges to terminal edges of the second pair of neurobands such that the first pair of neurobands can move relative to the second pair of neurobands along a portion of overlap.

22. The garment of claim 21, wherein a front panel of the garment comprises sections of variable elasticity.

23. The garment of claim 1, comprising a zipper on the front portion of the garment.
